(19) United States
(12) Patent Application Publication
(10) Pub. No.: US 2004/0247623 A1
(43) Pub. Date: Dec. 9, 2004

(54) Method and article for treatment of sensory neuron related disorders through transdermal application of botulinum toxin

(76) Inventor: Roger Cady, Springfield, MO (US)

Correspondence Address: HUSCH & EPPENBERGER, LLC, 190 CARONDELET PLAZA, SUITE 600, ST. LOUIS, MO, 63105-3441, US

(21) Appl. No.: 10/808,008

(22) Filed: Mar. 24, 2004

Related U.S. Application Data
(60) Provisional application No. 60/456,925, filed on Mar. 24, 2003.

(51) Int. Cl. A61K 38/43; A61K 39/08

(52) U.S. Cl. 424/239.1

(57) ABSTRACT

A method of treating migraine and other disorders associated with the release of certain neurotransmitters from sensory neurons includes the steps of preparing a medicament comprising Botulinum toxin type A as active-ingredient and applying the medicament transdermally to a human exhibiting symptoms associated with migraine or another sensory neuron related disorder. A pharmaceutical composition for transdermal application comprising Botulinum toxin type A as active-ingredient for administration to a human exhibiting symptoms of migraine or another sensory neuron related disorder is also provided.

### CROSS-REFERENCES

This application claims the priority of provisional application Ser. No. 60/456,925, filed Mar. 24, 2003.

### TECHNICAL FIELD OF THE INVENTION

The present invention relates generally to the treatment of migraine and other sensory neuron related disorders and, more particularly, to a method and article for treatment of migraine and other sensory neuron related disorders through transdermal application of Botulinum toxin type A.

### BACKGROUND OF THE INVENTION

A headache may be one of several different varieties, each of which has its own unique pain characteristics which differ dramatically. The types of headache include tension, sinus, cluster, rebound and migraine. Migraine is a particularly painful headache that recurs from time to time. The pain is quite severe and often the person with migraine must stay in bed. Dietary, emotional and environmental factors may trigger an attack. On average, migraine sufferers experience an attack per month. Attacks last from four to seventy-two hours. Of interest is that the incidence of migraine appears to be on the rise. Because of the severity and incidence of migraine, prescription medicines have been invented to provide relief.

Current theories on migraine suggest that the trigeminovascular system is a key contributor to migraine headaches. In a leading model, it is theorized that trigeminal afferents innervating meningeal vessels are activated during migraine. This activation results in the stimulation of afferents in the Opthalmic (V1) Branch of the Trigeminal Nerve. These afferents release a number of neuropeptides, including calcitonin gene-related peptide (“CGRP”). This release of CGRP results in vasodilation, neurogenic inflammation, and a decreased threshold for sensory activation of the involved trigeminal afferent. Therefore, the release of neuropeptides, including CGRP, is thought to be a key component to migraine. See Lassen, L H, Hadersley P A, Jacobsen V B, et al. CGRP may play a causative role in migraine. Cephalgia 2001:22:54-61.

Methods of controlling the release of CGRP and other neuropeptides are known. For example, certain serotonin agonists tend to reduce CGRP levels. Also, triptans, which stimulate the 5-HT₁ receptors appear to inhibit the release of CGRP. However, there is a continued need for other, more effective means of controlling the release of CGRP.

A number of recent studies have suggested that Botulinum toxin type A may provide a suitable treatment for migraine. However, these studies have not yet identified the means by which Botulinum toxin type A functions in the context of migraine. Botulinum toxin type A is a neurotoxin that inhibits neuromuscular conduction. It binds to receptor sites on motor nerve terminals, thereby blocking the release of acetylcholine into the neuromuscular junction. This mechanism produces a chemical muscle paralysis that is localized in nature. Botulinum toxin type A has become a routine treatment for severe muscle spasms in many neurological conditions, such as cerebral palsy, multiple sclerosis, traumatic brain injury and spinal cord injuries and is marketed under the brand name Botox® by Allergan, Inc. Botox® is administered exclusively via injection directly into the muscle for which relief is sought. The maximum dose of Botox® that is normally injected into a single, targeted muscle is 25 units (U) with a volume of 0.05 to 0.15 ml.

Based on the neuromuscular effect of Botulinum toxin type A, some practitioners have begun administration of Botulinum toxin type A to migraine sufferers via injection to muscles in the affected area. However, this approach requires a high degree of precision in making the injection and also limits any relief to a relatively small area. Furthermore, injections of Botulinum toxin type A generally result in temporary muscle paralysis.

While muscle pain is a component of migraine, the recent studies regarding Botulinum toxin type A have failed to demonstrate that the muscle paralysis induced by Botulinum toxin type A is the mechanism responsible for the toxin's effectiveness in the treatment of migraine. Other researchers have suggested that Botulinum toxin type A may have antinociceptive effects, and, in particular, may inhibit the release of neurotransmitters, including CGRP, from sensory neurons. See Duggan MJ, Quinn CP, Chaddock J. A., et al. Inhibition of release of neurotransmitters from rat dorsal root ganglia by a novel conjugate of a Clostridium botulinum toxin A endopeptidase fragment and erythrina cristagalli lectin. J Biol Chem 2002; 277:34846-34852; Welch M J, Purkiss J R, Foster K A. Sensitivity of embryonic rat dorsal ganglia neurons to Clostridium botulinum neurotoxins. Toxicon. 2000; 38:245-258. However, a truly efficacious method of administering Botulinum toxin type A in a manner which takes advantage of this sensory effect is needed.

The present invention is directed to addressing one or more of the identified problems and needs set forth above.

### SUMMARY OF THE INVENTION

An aspect of the present invention is to provide an improved method of treating migraine and other sensory neuron related disorders by the administration of Botulinum toxin.

Another aspect of the present invention is to provide an improved method of directly treating an area affected by migraine or other sensory neuron related disorders with Botulinum toxin type A and an associated apparatus for delivery of Botulinum toxin to the affected area.

In accordance with the above aspect of the invention, there is provided a use of Botulinum toxin type A in the manufacture of a medicament for transdermal administration to a human exhibiting symptoms of migraine or another a sensory neuron related disorder.

There is also provided a pharmaceutical composition for transdermal application comprising Botulinum toxin type A as active-ingredient for administration to a human exhibiting symptoms of migraine or another sensory neuron related disorder.

There is also provided a method of treating migraine that includes the steps of preparing a medicament comprising Botulinum toxin type A as active-ingredient and applying the medicament transdermally to a human exhibiting symptoms of migraine or another sensory neuron related disorder.

These aspects are merely illustrative of the innumerable aspects associated with the present invention and should not be deemed as limiting in any manner. These and other aspects, features and advantages of the present invention will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1 illustrates a schematic view of a transdermal patch for use with the present invention.

### DETAILED DESCRIPTION

Current theories relating to the cause of migraine suggest that migraine episodes are initiated by activation of the trigeminovascular system. Activation of trigeminal neurons elevates levels of CGRP. Subsequent reduction in CGRP levels is coincident with alleviation of the migraine, which suggests that CGRP is directly implicated in the mechanism of migraine. It is thought that Botulinum toxin type A directly inhibits the release of CGRP from trigeminal sensory neurons. However, the current practice of injecting Botulinum toxin type A, which is based on the neuromuscular effect of the neurotoxin rather than its sensory impact, involves injecting the neurotoxin into discreet muscles distant from the trigeminally innervated meningeal vessels thought to be involved in migraine.

Botulinum toxin type A has a sensory effect and, in particular, inhibits the release of neurotransmitters, including CGRP. The present invention relates to administering Botulinum toxin type A to migraine sufferers other than by injection, in particular, transdermal application. In particular, transdermal application of Botulinum toxin type A to an area affected by migraine or other sensory neuron related disorders is particularly advantageous. Transdermal application of Botulinum toxin type A requires far less precision in administration than an injection. Transdermal application of Botulinum toxin type A allows such application to the entire affected area. Transdermal application also allows administration of Botulinum toxin type A to a much larger area than is possible with an injection. Because the neurotoxin is applied to the skin and transported to the underlying trigeminal neurons rather than directly into the muscle, the temporary muscle paralysis associated with injecting Botulinum toxin type A is avoided.

Transdermal application of Botulinum toxin type A may be accomplished through any number of known methods. While exemplary methods and articles are described herein, the invention is not limited to any particular method or article for transdermal administration.

One transdermal application of Botulinum toxin type A involves preparation and administration of a topical cream. One embodiment of this application, includes reconstituting Botulinum toxin type A with preservative-free normal saline. The reconstituted toxin is then mixed in a syringe with a suitable base or carrier. The area to be treated is then cleansed with warm water, and the topical cream is massaged into the affected area.

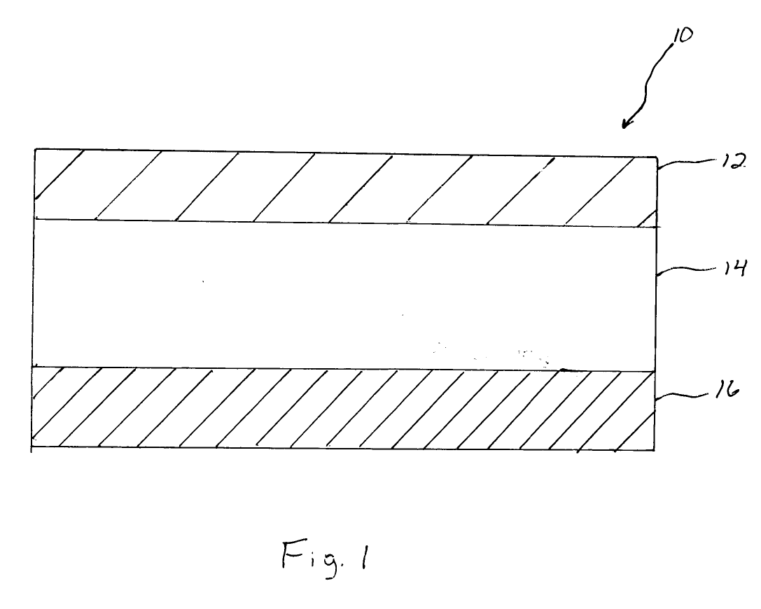

A suitable article for transdermal application of Botulinum toxin type A is incorporation of the neurotoxin into a transdermal patch, such as a methyl cellulase patch. Transdermal patches are known in the art. Examples of such patches are disclosed in U.S. Pat. Nos. 6,312,716 and 6,280,763, which are hereby incorporated by reference. An example of such a patch 10 is illustrated in FIG. 1. These patches are generally laminate in nature and include a backing layer 12, a reservoir layer 14 that contains the active ingredient, in this case Botulinum toxin type A, and a release layer 16.

A suitable method for transdermal administration of Botulinum toxin type A is electrophoresis.

The sensory effect of Botulinum toxin type A also lends itself to the treatment of diabetic neuropathy and other disorders that may be associated with the release of certain neurotransmitters from sensory neurons. Diabetic neuropathies are a family of nerve disorders caused by diabetes. Diabetes can damage nerves throughout a patient's body. These neuropathies result in numbness, pain and weakness in the hands, arms, feet and legs. While the precise cause of diabetic neuropathy remains unclear, researchers have identified a number of factors, including metabolic factors, such as high blood glucose, low insulin levels, and abnormal blood fat levels; neurovascular factors, leading to damage to the blood vessels that carry oxygen and nutrients to the nerves; autoimmune factors that cause inflammation in nerves; mechanical injury to nerves, such as carpal tunnel syndrome; inherited traits that increase susceptibility to nerve disease; and lifestyle factors such as smoking or alcohol use.

Transdermal application of Botulinum toxin type A to areas of a patient suffering from diabetic neuropathy inhibits the release of certain neurotransmitters in trigeminal neurons and results in an alleviation of the pain associated with the diabetic neuropathy. Botulinum toxin type A may be applied to a diabetic neuropathy patient in the same manner as described above in connection with the treatment of migraine using any number of known transdermal application methods.

The method will now be further illustrated with reference to the following non-limiting example.

### BIOLOGICAL EXAMPLE

One (1) vial of Botulinum toxin type A was reconstituted with 1 cc of preservative free normal saline and mixed in a syringe with 2 cc of PLO Gel (Pluronic Lecithin Organogel) containing 14 ml lecithin/Isopropyl Palmitate solution and 46 ml of 20% Pluronic F 127 solution. The affected area of a patient suffering from migraine was cleansed with warm water. Approximately 1.5 cc of the resulting topical cream was massaged into the frontalis, procerus, and temporal areas. Approximately 1 cc was massaged from the insertion to the body of the trapezium. Approximately 0.5 cc was massaged into the splenius area. The patient experienced relief from the migraine

Other objects, features and advantages of the present invention will be apparent to those skilled in the art. While preferred embodiments of the present invention have been illustrated and described, this has been by way of illustration and the invention should not be limited except as required by the scope of the appended claims and their equivalents.

What is claimed is:

1. A method of treating a sensory neuron related disorder, comprising the step of transdermally applying a pharmaceutically effective amount of Botulinum toxin type A to an affected area of a human exhibiting symptoms of the sensory neuron related disorder.

2. The method of treating a sensory neuron related disorder as set forth in claim 1, including the preliminary step of preparing a topical solution in which Botulinum toxin type A is the active ingredient.

3. The method of treating a sensory neuron related disorder as set forth in claim 2, wherein the step of preparing a topical solution further comprises the steps of:
reconstituting a pharmaceutically effective amount of Botulinum toxin type A with saline; and
mixing the reconstituted Botulinum toxin type A with a suitable base.

4. The method of treating a sensory neuron related disorder as set forth in claim 3, wherein the suitable base includes a pluronic lecithin organogel.

5. The method of treating a sensory neuron related disorder as set forth in claim 1, further comprising the step of providing a transdermal patch in which Botulinum toxin type A is the active ingredient, and wherein the step of transdermally applying the Botulinum toxin type A includes applying said transdermal patch at an affected area of the human.

6. The method of treating a sensory neuron related disorder as set forth in claim 5, wherein the transdermal patch comprises a backing layer, a reservoir layer containing the Botulinum toxin type A, and a release layer.

7. The method of treating a sensory neuron related disorder as set forth in claim 1, wherein the sensory neuron related disorder is migraine.

8. The method of treating a sensory neuron related disorder as set forth in claim 1, wherein the sensory neuron related disorder is diabetic neuropathy.

9. The method of treating a sensory neuron related disorder as set forth in claim 1, wherein the step of transdermally applying the Botulinum toxin type A comprises electrophoresis.

10. An article for the treatment of a sensory neuron related disorder comprising:
a backing layer;
a reservoir layer containing a pharmaceutically effective amount of Botulinum toxin type A as active ingredient therein; and
a release liner.

11. A topical solution for the treatment of a sensory neuron related disorder comprising:
a pharmaceutically effective amount of Botulinum toxin type A as active ingredient reconstituted in saline; and
a suitable base.

12. The topical solution for the treatment of a sensory neuron related disorder as set forth in claim 11, wherein the base comprises a pluronic lecithin organogel.

13. The topical solution for the treatment of a sensory neuron related disorder as set forth in claim 12, wherein the pluronic lecithin organogel contains a lecithin/isopropyl palmitate solution and a pluronic F127 solution.

14. A method of treating a sensory neuron related disorder, comprising the step of inhibiting the release of neuropeptides by the transdermal application of a pharmaceutically effective amount of Botulinum toxin type A to a human exhibiting symptoms of the sensory neuron related disorder.

15. The method of treating a sensory neuron related disorder as set forth in claim 14, wherein the neuropeptide inhibited is calcitonin gene-related peptide.

16. A method of treating migraine, comprising the steps of:
reconstituting a pharmaceutically effective amount of Botulinum toxin type A with saline;
mixing the reconstituted Botulinum toxin type A with a base including a pluronic lecithin organogel; and
transdermally applying the topical cream to an affected area of a human exhibiting symptoms of migraine.
